(19) United States
(12) Patent Application Publication
(10) Pub. No.: US 2005/0197620 A1
(43) Pub. Date: Sep. 8, 2005

(54) Safety trocar device

(76) Inventor: Fung-Chao Tu, Chiao City (TW)

Correspondence Address: TROXELL LAW OFFICE PLLC;SUITE 1404, 5205 LEESBURG PIKE, FALLS CHURCH, VA, 22041, US

(21) Appl. No.: 10/790,043

(22) Filed: Mar. 2, 2004

(51) Int. Cl. A61M 37/00

(52) U.S. Cl. 604/26; 604/158; 606/167

(57) ABSTRACT

A safety trocar device designed for safe abdominal endoscope operation, including a trocar assembly and a bracket. The trocar assembly includes a sleeve, a trocar and an insufflation needle. A bottom end of the trocar assembly has a thrust section. In use, the trocar assembly is mounted on a suspension arm of the bracket and the bracket is fixed on an operation table. The bracket has an elevation arm under the suspension arm for hooking the abdominal wall of a patient. In operation, an operator continuously turns the trocar assembly with one hand and the abdominal wall is gradually elevated by the elevation arm, whereby the thrust section penetrates through the abdominal wall. Accordingly, the trocar assembly will only drill through the elevated abdominal wall without touching the organs in the abdominal cavity. The trocar assembly is provided with penetration indicator for the operator to judge whether the trocar assembly has truly penetrated through the abdominal wall.

### BACKGROUND OF THE INVENTION

The present invention is related to an abdominal endoscope device, and more particularly to a safety trocar device including a trocar assembly and a bracket. The safety trocar device ensures safety during abdomen entry operation and prevents the abdominal organs and vessels of the patient from being injured.

It is a trend to use abdominal endoscope in abdominal operation to minimize injury of human body and speed recovery. The first step of the operation is abdomen entry. In this step, a sharp implement such as insufflation needle or a trocar is thrust into the abdominal cavity. This step is the most dangerous part of the operation. This is because that at this time, it is most possible to injure the vessels or organs in the abdominal cavity. Many new inventions and improved products have been developed for solving this problem. However, none of them is absolutely secure. The most widely used measure for abdomen entry is to elevate the abdominal wall with one hand and then penetrate the elevated abdominal wall with an insufflation needle 12 or a trocar 14 as shown in FIG. 1. However, it is hard to accurately control the strength of the hand and the depth of the thrust. An surgeon can be skilled in these only by means of repeated learning and accumulated experience. In addition, the danger in abdomen entry is also caused by failure of the safety protection function of the implement or not totally evitable tenting.

### SUMMARY OF THE INVENTION

It is therefore a primary object of the present invention to provide a trocar assembly by which during penetration of abdominal wall, the danger of injury of great vessels or organs in the abdominal cavity can be avoided.

It is a further object of the present invention to provide the above trocar assembly which has penetration indicators. By means of the penetration indicators, an operator can accurately know whether the tip of the trocar assembly has entered the abdominal cavity.

It is still a further object of the present invention to provide the above trocar assembly which enables an operator to perform various tests for judging whether the trocar assembly has truly entered the abdominal cavity.

It is still a further object of the present invention to provide an operation-used bracket. The bracket is co-used with the above trocar assembly to form a trocar device. By means of the bracket, the abdominal wall of a patient can be gradually elevated for the trocar assembly to penetrate.

The present invention can be best understood through the following description and accompanying drawings wherein:

### BRIEF DESCRIPTION OF THE DRAWINGS

FIG. 1 shows the conventional penetration operation of abdominal wall;

FIG. 2 is a perspective assembled partially sectional view of the trocar assembly of a preferred embodiment of the present invention;

FIG. 3 is a perspective exploded view according to FIG. 2;

FIG. 4 is a perspective sectional view of the sleeve of FIG. 2;

FIG. 5 is a longitudinal sectional view according to FIG. 2;

FIG. 6 is a perspective view of the bracket of the preferred embodiment of the present invention;

FIG. 7 shows a state of a patient prior to the operation;

FIG. 8 shows that the present invention is mounted on an operation table;

FIG. 9 shows that an operation operates the present invention;

FIGS. 10 to 15 show the procedure of the penetration of the present invention through the abdominal wall; and

FIG. 16 shows a state of completion of the penetration operation.

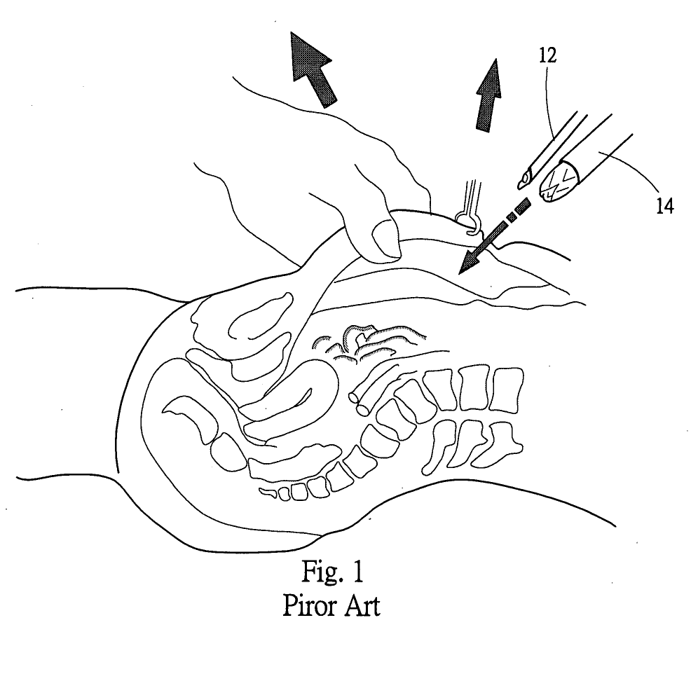

### DETAILED DESCRIPTION OF THE PREFERRED EMBODIMENTS

The trocar device of the present invention includes a trocar assembly and a bracket on which the trocar assembly is mounted. The tip of the trocar is fixed on a safe point. The abdominal wall is gradually elevated by means of the bracket. The trocar penetrates the abdominal wall and gradually expands the wound. Then the bracket is removed and the abdominal cavity is insufflated for abdominal endoscope operation.

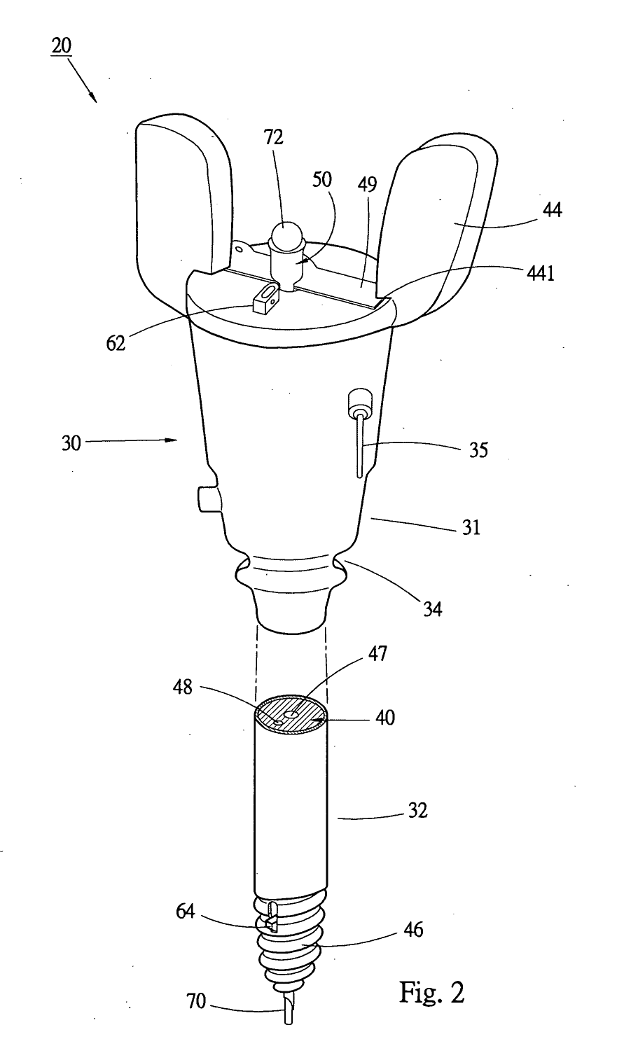

Please refer to FIGS. 2 to 5. The trocar assembly 20 includes a sleeve 30, a trocar 40 and an insufflation needle 50. These three parts can be fast assembled and disassembled.

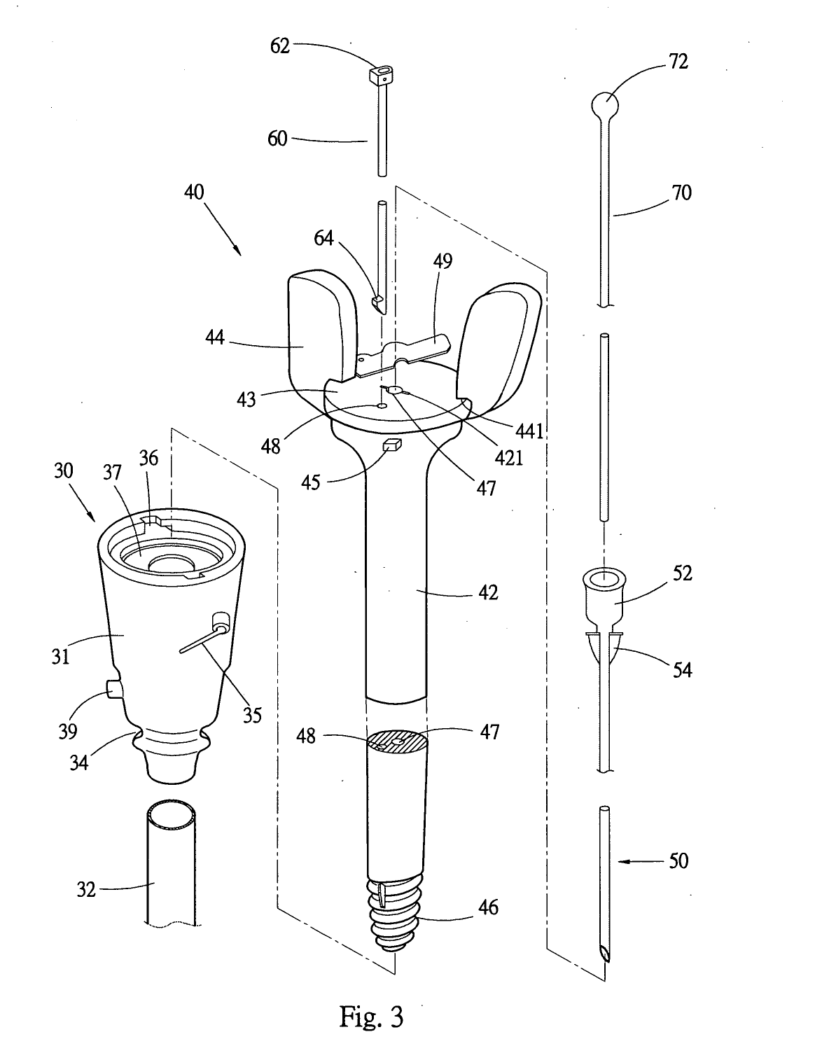

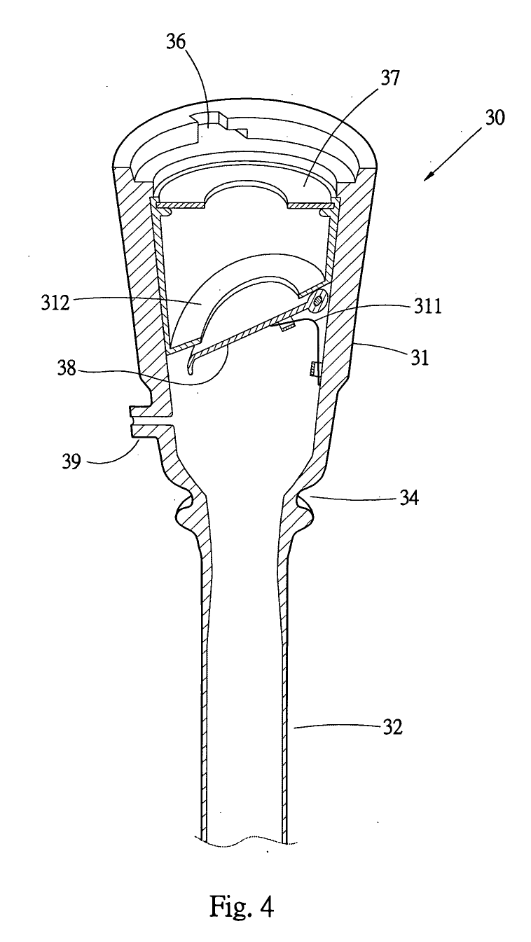

Referring to FIGS. 3 and 4, the sleeve 30 is made of plastic or metal material, having an upper section 31 and a relatively slender tubular lower section 32. The outer circumference of an adjoining section between the upper and lower sections is annularly grooved to form a clamped section 34 for the bracket to fix the sleeve. The opening of the upper section 31 is formed with two latch notches 36 spaced from each other by a certain distance. Several airtight members are disposed under the latch notches. The airtight members include a diaphragm ring 37 and a petal valve 38 positioned on lower side of the diaphragm ring 37. The petal valve 38 is pivotally rotatably disposed in the sleeve 30 with exhaustion (airtight) function. The pivot section of the petal valve 38 is connected with a crank 35 which is pivotally disposed on outer circumference of the sleeve for an operator to control from outer side. In addition, a resilient member 311 such as an L-shaped steel filament is connected between bottom face of the petal valve and inner circumference of the upper section 31. In normal state, the resilient member 311 serves to resiliently angularly displace the petal valve 38 to airtightly close on a petal ring 312 formed on inner circumference of the upper section 31. In addition, a passage 39 is formed on the circumference of the sleeve 30 for communicating with the interior thereof. The passage 39 serves to connect with a stopcock for controlling entry of the gas into the sleeve.

The trocar 40 is metal-made and has a circular rod-shaped body section 42. A tray section 43 is disposed on top end of the body section. A pair of rotary switches 44 are disposed on the top end of the body section for manual turning. A gap 441 is defined between the bottom of inner side of each rotary switch 44 and the top face of the tray section 43. Two engaging bodies 45 are disposed on the circumference of the body section 42 at certain intervals. The bottom end of the body section is a conic thrust section 46. The circumference of the thrust section is formed with coarse thread. In addition, a fine axial central tunnel 47 and a fine axial lateral tunnel 48 are formed in the body section. Both the tunnels pass through the body section 42 from top end to bottom end thereof. The central tunnel 47 is positioned at the center of the trocar so that the bottom end of the central tunnel 47 right passes through the tip of the thrust section 46. The lateral tunnel 48 is adjacent to one side of the trocar so that the bottom end of the lateral tunnel passes through the circumference of the thrust section.

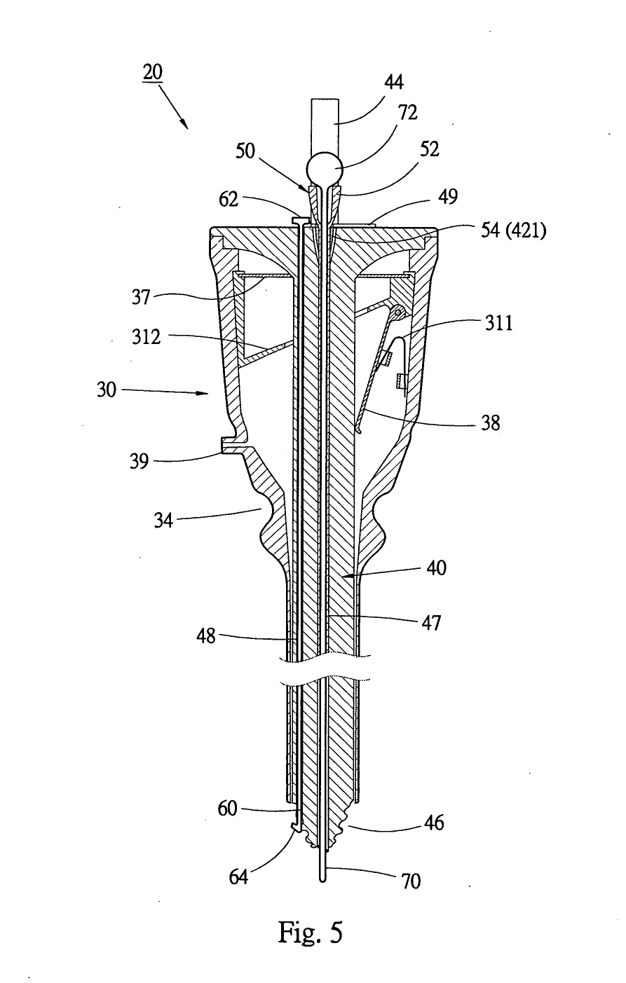

The insufflation needle 50 is a slender tube body having an injection opening 52 at top end and two wing-shaped locating sections 54 positioned under the injection opening 52. The insufflation needle 50 is fitted in the central tunnel 47 as shown in FIG. 5. The two locating sections 54 are inserted in two insertion splits 421 formed on top face of the body section 42 of the trocar 40 to locate the insufflation needle 50. After located, an oblique cut section of the tip of bottom end of the insufflation needle 50 right protrudes from the tip of the thrust section 46 of the trocar 40. A fixing member 49 which is a plate body is pivotally disposed on top end of the trocar 40 and displaceable between a latched position and an unlatched position. When the fixing member 49 is moved to the latched position, the fixing member is inserted into the gaps 441 of the trocar to press the locating sections 54 of the insufflation needle 50 for locating the insufflation needle 50 in the central tunnel. When the fixing member 49 is moved out of the gaps 441, the insufflation needle 50 is no longer pressed and can be taken out.

A first indicator member 60 is fitted in the lateral tunnel 48. The first indicator member 60 is a slender metal rod-shaped probe. Top end of the probe has an indicating section 62, while bottom end of the probe has an obtuse probe head 64. The length of the probe is 0.5 cm longer than the length of the lateral tunnel. Therefore, the probe head 64 extends out of the trocar 40 and protrudes from the circumference of the thrust section 46. When the probe is not touched by external force, the probe will fall due to gravity, whereby the indicating section 62 will abut against the top end of the body section 42 of the trocar.

A second indicator member 70 is fitted in the insufflation needle 50. The second indicator member 70 is a slender rod-shaped indicator needle. The length of the indicator needle 70 is 0.5 cm longer than the length of the insufflation needle 50. Top end of the indicator needle has an indicating section 72. The indicator needle has a round and dull bottom end. When the indicator needle is not touched by external force, the indicator needle will fall due to gravity, whereby the bottom end of the indicator needle will protrude from the thrust section 46 of the trocar and the indicating section 72 will fall on the top end of the insufflation needle 50.

After the insufflation needle 50, probe 60 and the indicator needle 70 are all installed in the trocar 40, the trocar 40 is fitted into the sleeve 30. In installation, the crank 35 is turned to move the petal valve 38 downward away from the petal ring 312, whereby the trocar 40 can be mounted into the sleeve 30. After the trocar is fitted into the sleeve, the tray section 43 abuts against the top end of the sleeve. The two engaging bodies 45 of the trocar are latched in the latch notches 36 of the sleeve. The trocar is tightly fitted in the lower section 32 of the sleeve with the thrust section 46 just protruding from the bottom end of the sleeve 30. Also, the probe 60 and the indicator needle 70 protrude from the bottom end of the trocar. Accordingly, the trocar assembly 20 of the present invention is completed.

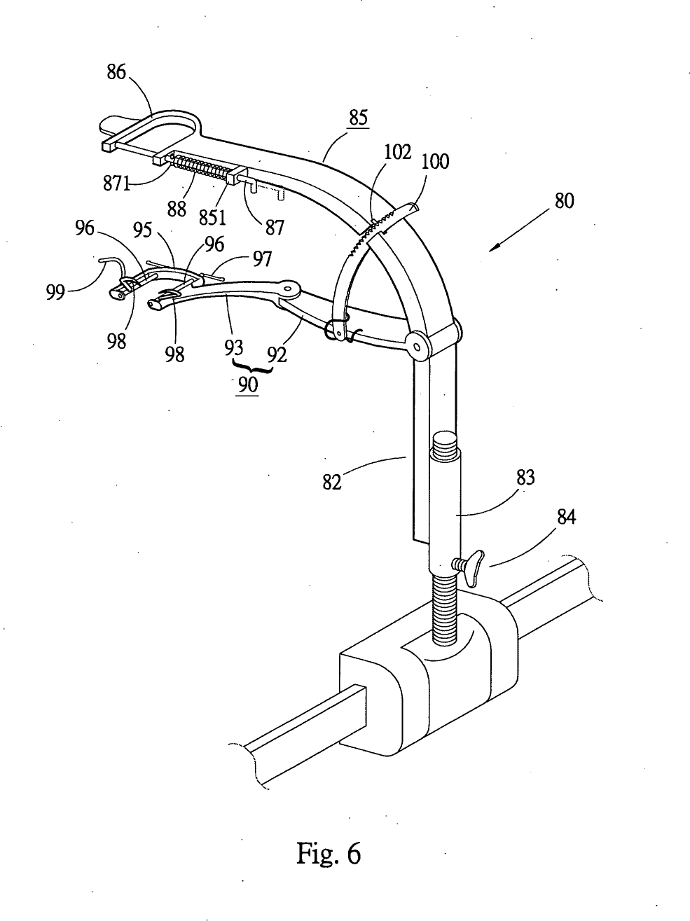

In abdominal endoscope operation, the trocar assembly is mounted on the bracket. Referring to FIG. 6, the bracket 80 includes a bracket body 82. A mounting section 83 is disposed at bottom end of the bracket body 82. The mounting section 83 is a collar. A bolt 84 is screwed through the wall of the collar 83.

The bracket 80 further includes a suspension arm 85 outward extending from the bracket body 82 as a cantilever. One end of the suspension arm 85 is formed with a C-shaped fitting mouth 86. Two lugs 851 are disposed on the suspension arm 85. The lugs are positioned on the same side as the opening of the fitting mouth 86. A pin member 87 is fitted through the lugs 851. One end of a spring 88 abuts against a projecting section 871 of the pinmember 87, while the other end of the spring 88 abuts against a lug 851 for pushing the pin member 87 toward the fitting mouth 86 to close the opening thereof.

The bracket further includes an elevation arm 90 having a first arm body 92 and a second arm body 93 pivotally connected with each other. The arm bodies 92, 93 are such pivotally connected that the second arm body 93 can be horizontally rotated relative to the first arm body 92. One end (free end of the first arm body) of the elevation arm 90 is pivotally connected on the bracket body 82 and up and down angularly displaceable to provide a leverage effect. The elevation arm is positioned right under the suspension arm 85 and the distance between the elevation arm and the suspension arm is adjustable. A free end of the elevation arm (free end of the second arm body) is formed with a C-shaped pulling/lifting section 95. Two rotary shafts 96 are pivotally disposed on two sides of the pulling/lifting section 95. The two rotary shafts 96 are parallelly spaced from each other by a certain distance. One end of each rotary shaft 96 is fixedly connected with a shift switch 97 for rotating the rotary shaft. Two hook ears 98 are respectively fixedly connected on the two rotary shafts 96. When rotating the rotary shafts, the hook ears 98 are moved downward to hook a body or moved upward to release the body.

In addition, a locating member such as a ratchet plate 100 is disposed on the elevation arm 90. One end of the ratchet plate 100 is pivotally connected on the elevation arm, while the other end of the ratchet plate upward extends through a slot 102 formed on the bracket body 82. A skipping ratchet (not shown) is disposed at the slot for engaging with the ratchets of the ratchet plate. When the elevation arm 90 is gradually elevated toward the suspension arm 85, the ratchet plate 100 is engaged with the ratchet to keep the elevation arm at a fixed height without descending. The engagement between the ratchet plate and the ratchet will not be described in detail.

The operation manner of the present invention will be described hereinafter.

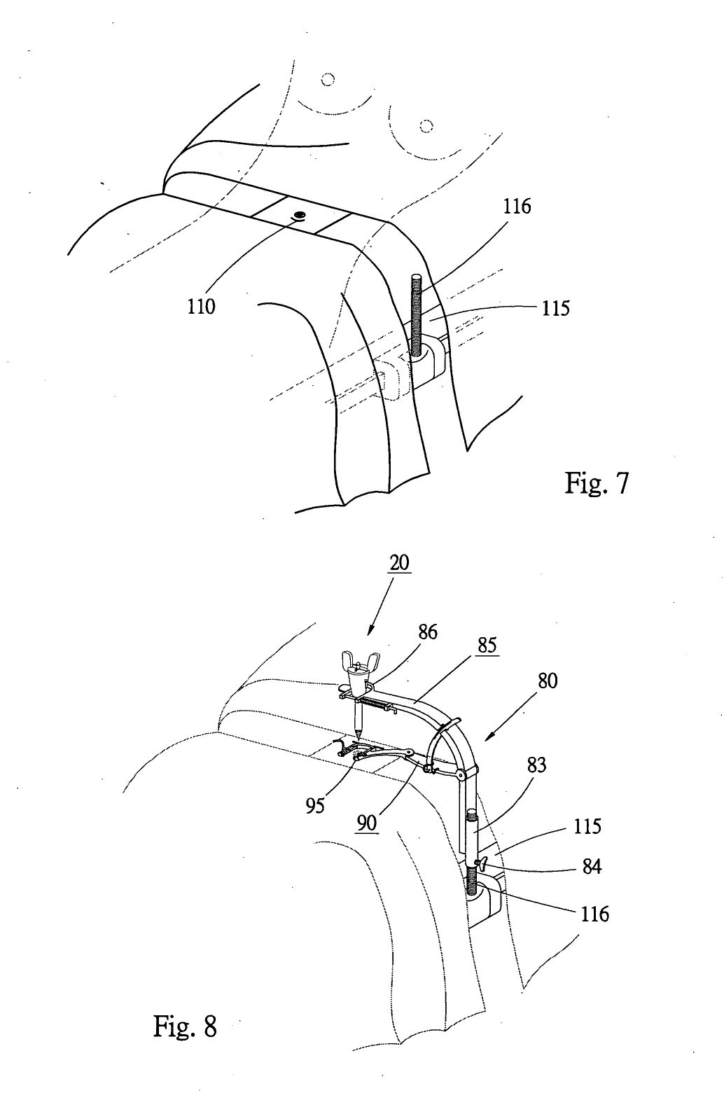

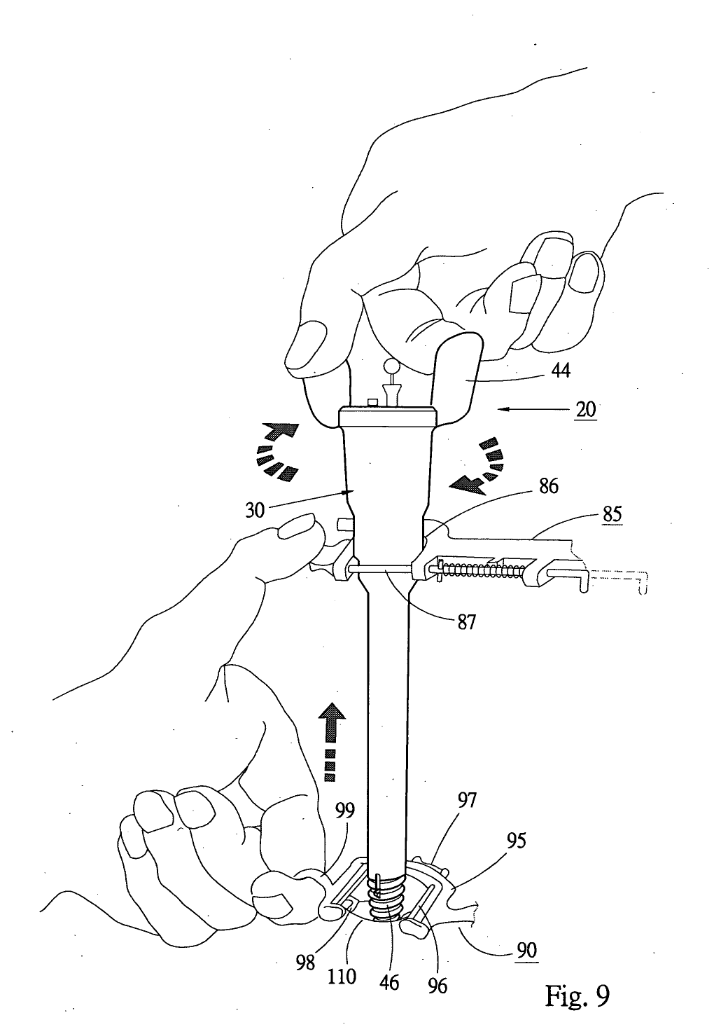

A patient read for the operation is incised with a transverse or semicircular small cut 110 on lower side of the navel. The small cut 110 is about 1 cm long as shown in FIG. 7.

The disinfected bracket 80 is fixed on an edge of an operation table 115 as shown in FIG. 8. The collar 83 is fitted on a column 116 of the operation table. After the bracket is adjusted to a certain height, the bolt 84 is tightened to fix the bracket. After mounted, the pulling/lifting section 95 of the free end of the elevation arm 90 is positioned right on the plane of the skin of the cut 110 under the navel. Then, an operator turns the hook ears 98 downward to hook two sides of the cut 110.

The pin member 87 on the suspension arm is pulled to open the opening of the fitting mouth 86. Then the trocar assembly 20 is installed into the fitting mouth 86. The clamped section 34 of the sleeve 30 is fitted in the fitting mouth 86. After the pin member 87 is released from the pulling force, the pin member closes the fitting mouth 86 to hold the clamped section 34. At this time, the installation of the trocar assembly is completed. The trocar assembly can be rotated within the fitting mouth. The tip of lower end of the trocar assembly is about 4 cm spaced from the pulling/lifting section 95 of the elevation arm as shown in FIG. 8.

After the above procedure is completed, the index finger of one hand (such as left hand) of the operator pulls a pull hook 99 of the free end of the elevation arm 90 to elevate the elevation arm toward the suspension arm 85. At this time, the thrust section 46 of bottom end of the trocar 40 extends through the pulling/lifting section 95 into the cut 110. Then, one hand (such as right hand) turns the rotary switches 44 of the trocar to make the thrust section of the trocar drill the abdominal wall. With the abdominal wall gradually elevated by the elevation arm 90, the trocar can continuously gradually drill through the abdominal wall.

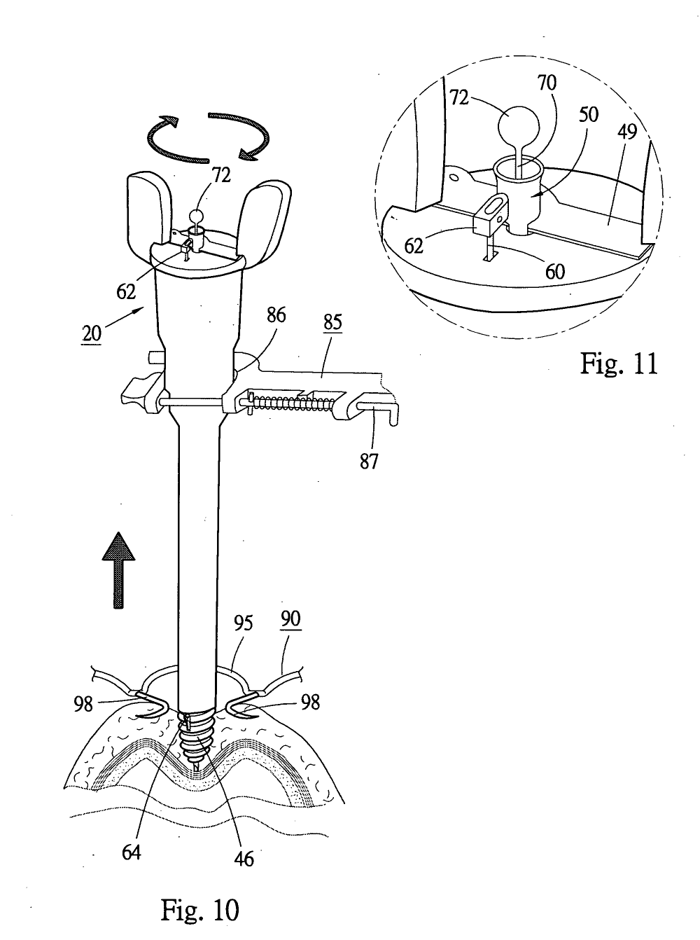

When the thrust section 46 of the bottom end of the trocar 40 contacts with the abdominal wall, as shown in FIG. 10, a resistant force is exerted onto the bottom ends of the probe 60 and the indicator needle 70 so that the probe 60 and the indicator needle 70 are moved upward. At this time, the indicating sections 62, 72 of top ends of the probe 60 and the indicator needle 70 are both lifted as shown in FIG. 11. Accordingly, the operator can judge that the trocar assembly has not yet penetrated through the abdominal wall.

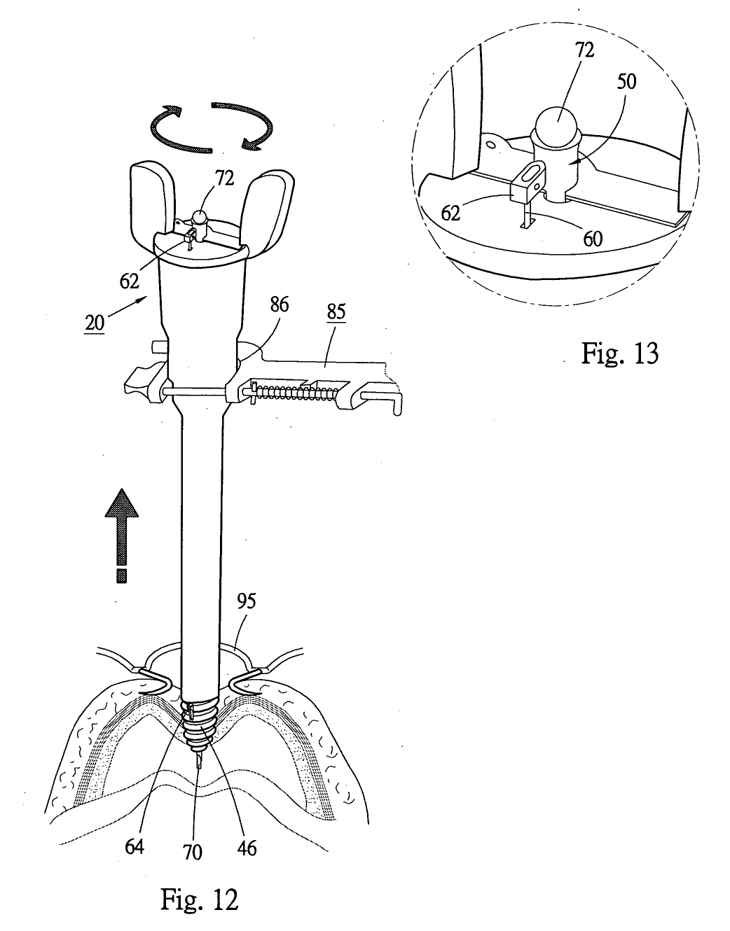

Once the tip of the thrust section 46 of the trocar penetrates through the abdominal wall and enters the abdominal cavity, as shown in FIG. 12, the bottom end of the indicator needle 70 is free from any stop and falls due to gravity. At this time, the indicating section 72 of the indicator needle 70 descends onto the top end of the trocar 50 as shown in FIG. 13. Accordingly, the operator can judge that the tip of the trocar assembly has already entered the abdominal cavity. Moreover, as shown in FIG. 12, the abdominal wall is lifted by the elevation arm and spaced from the organs in the abdominal cavity by a certain distance. Therefore, when the trocar enters the abdominal cavity, the organs or great vessels will not be injured.

In the state of FIG. 12, various tests can be performed for the operator to judge whether the trocar assembly has truly entered the abdominal cavity. For example, the indicator needle 70 can be taken out from the insufflation needle 50. Instead, a syringe filled with water is inserted into the injection opening to inject water through the insufflation needle into the abdominal cavity. Alternatively, the air or material in the abdominal cavity can be sucked out by the insufflation needle. By means of these tests, the operator can know whether the trocar assembly has truly entered the abdominal cavity so as to ensure safety. In few conditions, the organs of the patient may adhere to the abdominal wall. Therefore, by means of the test in this stage, the operator can realize whether the thrust section touches the organs adhering to the abdominal wall. If so, the thrust operation should be immediately stopped so as to avoid injury or further injury.

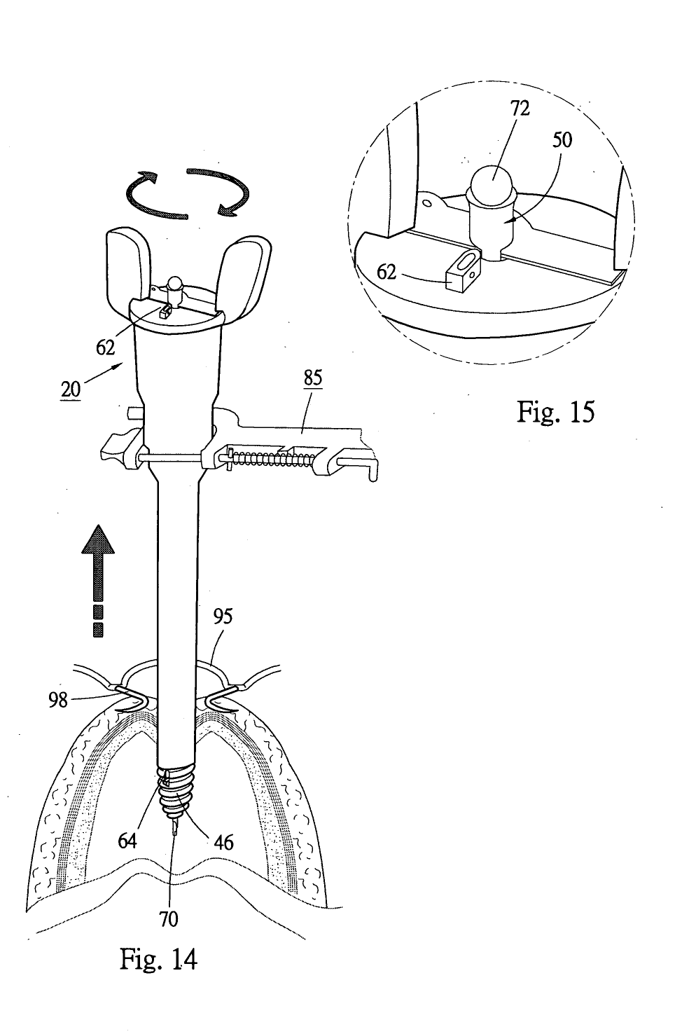

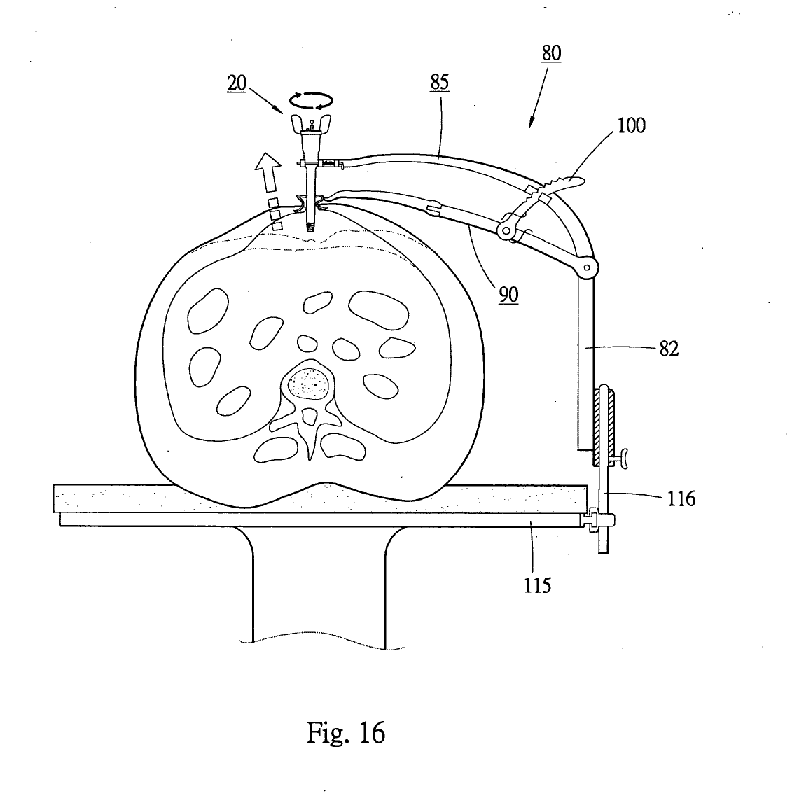

After the operation safely goes to the state of FIG. 12, the operator further performs the abdomen entry operation to make the thrust section 46 totally penetrate through the abdominal wall into the abdominal cavity as shown in FIGS. 14 and 15. At this time, the probe head 64 of the bottom end of the probe 60 is not stopped so that the indicating section 62 of the top end of the probe 60 descends. This means the entire thrust section has entered the abdominal cavity in a state as shown in FIG. 16.

At this time, the thrust operation of the abdominal wall has been completed. The operator can separate the bracket from the trocar assembly and remove the bracket. Then the trocar 40 with the insufflation needle, the probe and the indicator needle is taken out from the sleeve 30. Then the abdominal cavity is inflated through the passage 39. Then an abdominal endoscope or a surgical implement can be extended into the abdominal cavity through the sleeve for operation.

With the trocar device of the present invention, the conventional measure that a sharp insufflation needle or a trocar is manually thrust through the elevated abdominal wall is not used Instead, the not omissible and most dangerous sharp tip of the insufflation needle or trocar is fixed in a safe fixed point outside the abdominal wall. The abdominal wall is gradually elevated under control and the trocar assembly is turned to gradually penetrate the abdominal wall. Accordingly, the main vessels and organs in the abdominal cavity are not moved so that the possibility of injury of the vessels and organs is eliminated. Furthermore, the insufflation needle and the trocar successively penetrate the abdominal wall by way of screwing and the cut is gradually expanded. Therefore, the tenting can be effectively avoided and the left penetration wound is minimized. In addition, the insufflation needle, the trocar and the sleeve enter the abdominal cavity at the same time so that the operation procedure is simplified. The two indicating sections positioned at the center and the edge of the trocar enable the operation to accurately know that the tip or entire sleeve has entered the abdominal cavity. Also, the insufflation needle at the center enables the operation to perform various tests for realizing whether the trocar assembly has truly entered the abdominal cavity.

The above embodiments are only used to illustrate the present invention, not intended to limit the scope thereof. Many modifications of the above embodiments can be made without departing from the spirit of the present invention.

1. A trocar assembly comprising:
a sleeve having an upper section and a relatively slender tubular lower section, at least one airtight member being disposed in the upper section;
a trocar having a rod-shaped body section, a bottom end of the body section being a conic thrust section, a circumference of the thrust section being spiraled; a first fine elongated tunnel being axially formed through the body section from top end to bottom end thereof; the trocar being fitted through the sleeve with the thrust section protruding from a bottom end of the sleeve; and
an insufflation needle which is a slender tube body fitted in the first tunnel of the trocar, a bottom end of the insufflation needle being positioned at the thrust section.

2. The trocar assembly as claimed in claim 1, further comprising an indicator needle which is a slender rod body, the indicator needle having a length longer than the length of the insufflation needle by a certain value; the indicator needle being fitted through the insufflation needle and movable within the insufflation needle, when the indicator needle is not touched by external force, the indicator needle falling due to gravity, whereby a top end of the indicator needle falls on the top end of the insufflation needle, while a bottom end of the indicator needle protrudes from the thrust section.

3. The trocar assembly as claimed in claim 2, wherein a second fine elongated tunnel is formed through the body section of the trocar from top end to bottom end thereof; the trocar assembly further comprising a probe, the probe having a length longer than the length of the second tunnel, the probe being fitted through the second tunnel and movable along the second tunnel, in normal state, the probe falling due to gravity, whereby a top end of the probe is located on the top end of the body section, while a bottom end of the probe protrudes from the bottom end of the trocar.

4. The trocar assembly as claimed in claim 3, wherein the first tunnel is positioned at the center of the body section of the trocar and the bottom end of the indicator needle right protrudes from the tip of the thrust section; the second tunnel being positioned on one side of the body section, the bottom end of the probe protruding from a circumference of the thrust section.

5. The trocar assembly as claimed in claim 3, wherein each of the top ends of the indicator needle and the probe has an indicating section.

6. The trocar assembly as claimed in claim 3, wherein the bottom end of the probe is an obtuse probe head.

7. The trocar assembly as claimed in claim 1, wherein a second fine elongated tunnel is formed through the body section of the trocar from top end to bottom end thereof; the trocar assembly further comprising a probe, the probe having a length longer than the length of the second tunnel, the probe being fitted through the second tunnel and movable along the second tunnel, in normal state, the probe falling due to gravity, whereby a top end of the probe is located on the top end of the body section, while a bottom end of the probe protrudes from the bottom end of the trocar.

8. The trocar assembly as claimed in claim 1, wherein a clamped section is formed on outer circumference of the sleeve for holding.

9. The trocar assembly as claimed in claim 1, wherein a tray section is disposed at top end of the body section of the trocar, whereby when the trocar is fitted through the sleeve, the tray section abuts against the top end of the sleeve.

10. The trocar assembly as claimed in claim 1, wherein at least one latch notch is formed at the opening of the upper section of the sleeve; at least one engaging body being formed at the top end of the body section of the trocar, whereby the engaging body can be engaged in the latch notch.

11. The trocar assembly as claimed in claim 1, further comprising a fixing member disposed on top end of the body section and displaceable between a latched position and an unlatched position, whereby when the fixing member is moved to the latched position, the fixing member is latched with the top end of the insufflation needle.

12. The trocar assembly as claimed in claim 1, wherein at least one insertion split is formed on top face of the body section of the trocar and at least one locating section is disposed at top end of the insufflation needle, whereby when the insufflation needle is fitted in the trocar, the locating section is inserted in the insertion split.

13. The trocar assembly as claimed in claim 12, further comprising a fixing member disposed on top end of the body section and displaceable between a latched position and an unlatched position, whereby when the fixing member is moved to the latched position, the fixing member is latched with the top end of the insufflation needle.

14. The trocar assembly as claimed in claim 13, wherein a gap is formed on the top end of the body section of the trocar and the fixing member is a plate body, one end of the plate body being pivotally rotatably connected with the top end of the trocar, whereby when the fixing member is moved to the latched position, the fixing member is inserted into the gap.

15. The trocar assembly as claimed in claim 1, wherein a passage is formed on outer circumference of the sleeve for communicating with the interior thereof.

16. The trocar assembly as claimed in claim 1, further comprising a rotary switch disposed on the top end of the trocar assembly.

17. The trocar assembly as claimed in claim 16, wherein the rotary switch is disposed on the top end of the body section of the trocar and protrudes from the sleeve.

18. A trocar assembly comprising:
a sleeve having an upper section and a relatively slender tubular lower section; at least one airtight member being disposed in the upper section;
a trocar having a rod-shaped body section, a bottom end of the body section being formed with a thrust section, a first fine elongated tunnel being axially formed through the body section from top end to bottom end thereof, the trocar being fitted through the sleeve with the thrust section protruding from a bottom end of the sleeve; and
a first slender indicator member having a length longer than the length of the first tunnel, the first indicator member being fitted through the first tunnel and movable within the first tunnel, in normal state, the first indicator member falling due to gravity, whereby a top end of the first indicator member is positioned on the top end of the trocar, while a bottom end of the first indicator member protrudes from the first tunnel.

19. The trocar assembly as claimed in claim 18, wherein the bottom end of the first tunnel is positioned right at the center of the thrust section.

20. The trocar assembly as claimed in claim 18, wherein the bottom end of the first tunnel is positioned on the circumference of the thrust section.

21. The trocar assembly as claimed in claim 19, wherein a second fine elongated tunnel is formed through the body section from top end to bottom end thereof, a bottom end of the second tunnel being positioned on the circumference of the thrust section; the trocar assembly further comprising a second indicator member having a length longer than the length of the second tunnel, the second indicator member being disposed in the second tunnel and movable within the second tunnel, in normal state, the second indicator member falling due to gravity, whereby a top end of the second indicator member is positioned on the top end of the trocar, while a bottom end of the second indicator member protrudes from the second tunnel.

22. The trocar assembly as claimed in claim 21, wherein each of the top ends of the indicator members has an indicating section.

23. The trocar assembly as claimed in claim 21, further comprising an insufflation needle which is a slender tube body fitted in the first tunnel, a bottom end of the insufflation needle being positioned at the thrust section; the first indicator member being fitted through the insufflation needle and movable within the insufflation needle.

24. A bracket co-used with a trocar assembly, comprising:
a bracket body, a mounting section being disposed at bottom end of the bracket body for connecting with an operation table;
a suspension arm connected with the bracket body and outward transversely extending from the bracket body as a cantilever;
a fitting mouth being disposed at a free end of the suspension arm; and
an elevation arm, one end of the elevation arm being pivotally connected on the bracket body and up and down displaceable to change height, the elevation arm being positioned under the suspension arm; a pulling/lifting section being disposed at a free end of the suspension arm under the fitting mouth; two hook ears being disposed on the pulling/lifting section for hooking a body, the hook ears being spaced from each other by a certain distance.

25. The bracket as claimed in claim 24, wherein the fitting mouth of the suspension arm is C-shaped; the bracket further comprising a pin member which unlatchably closes an opening of the fitting mouth.

26. The bracket as claimed in claim 25, wherein two lugs are disposed on the suspension arm on the same side as the opening of the fitting mouth; the pin member being fitted through the lugs and slidable between the lugs, when the pin member is slid toward the fitting mouth, the pin member closing the opening of the fitting mouth; a resilient member being disposed between the pin member and the suspension arm, whereby in normal state; the pin member is forced by the resilient member to close the fitting mouth.

27. The bracket as claimed in claim 26, wherein the resilient member is a spring fitted on the pin member, one end of the spring abutting one lug, while the other end of the spring abutting against the pin member.

28. The bracket as claimed in claim 24, wherein two rotary shafts are disposed on the pulling/lifting section of the elevation arm, the two rotary shafts are parallelly spaced from each other by a certain distance; the hook ears being respectively fixedly connected with the two rotary shafts.

29. The bracket as claimed in claim 28, wherein the pulling/lifting section is C-shaped and the rotary shafts are pivotally disposed on two sides of the pulling/lifting section.

30. The bracket as claimed in claim 29, wherein one end of each rotary shaft is fixedly connected with a shift switch for manually rotating the rotary shaft.

31. The bracket as claimed in claim 24, wherein one end of the elevation arm is pivotally connected with the bracket body, whereby the elevation arm can be up and down angularly displaced to change height.

32. The bracket as claimed in claim 31, wherein the elevation arm has a first arm body and a second arm body pivotally connected with each other, the second arm body being transversely rotatable relative to the first arm body; a free end of the first arm body of the elevation arm being pivotally connected on the bracket body; the pulling/lifting section being disposed at a free end of the second arm body.

33. The bracket as claimed in claim 24, wherein the mounting section is a collar; further comprising a bolt being screwed through a circumferential wall of the collar.

34. The bracket as claimed in claim 24, further comprising a locating member disposed between the elevation arm and the bracket body, whereby when the elevation arm is moved upward, the locating member serves to fix the elevation arm at a fixed height.

35. The bracket as claimed in claim 34, wherein a slot is formed on the suspension arm and at least one ratchet is disposed at the slot; the locating member being a ratchet plate, one end of the ratchet plate being connected with the elevation arm, while the other end of the ratchet plate extending through the slot to engage with the ratchet.

36. A safety trocar device comprising a trocar assembly and a bracket, wherein:
the trocar assembly including:
a sleeve;
a trocar having a rod-shaped body section, a bottom end of the body section being a conic thrust section; a first fine elongated tunnel being axially formed through the body section from top end to bottom end thereof, the trocar being fitted through the sleeve with the thrust section protruding from a bottom end of the sleeve; and
a slender member fitted through the first tunnel; and
the bracket includes:
a bracket body having a mounting section for connecting with an operation table;
a suspension arm connected with the bracket body and outward extending from the bracket body as a cantilever, a fitting mouth being disposed at a free end of the suspension arm; and
an elevation arm, one end of the elevation arm being pivotally connected on the bracket body and up and down displaceable to change height, the elevation arm being positioned under the suspension arm; a pulling/lifting section being disposed at a free end of the suspension arm under the fitting mouth, the pulling/lifting section serving to hook a body;
the trocar assembly being detachably mounted in the fitting mouth of the bracket and manually rotatable.

37. The safety trocar device as claimed in claim 36, wherein the slender member is an indicator member having a length longer than the length of the first tunnel, the indicator member being fitted through the first tunnel and movable within the first tunnel, in normal state, the indicator member falling due to gravity, whereby a top end of the indicator member is positioned on the top end of the trocar, while a bottom end of the indicator member protrudes from the first tunnel.

38. The safety trocar device as claimed in claim 36, wherein the slender member is a tubular insufflation needle.

39. The safety trocar device as claimed in claim 38, further comprising a first slender indicator member having a length longer than the length of the insufflation needle, the indicator member being fitted through the insufflation needle and movable within it, in normal state, the indicator member falling due to gravity, whereby a top end of the indicator member is positioned on the top end of the trocar assembly, while a bottom end of the indicator member protrudes from the first tunnel.

40. The safety trocar device as claimed in claim 39, wherein a second fine elongated tunnel is formed through the body section of the trocar from top end to bottom end thereof; further comprising a second indicator member having a length longer than the length of the second tunnel, the second indicator member being disposed in the second tunnel and movable within the second tunnel, in normal state, the second indicator member falling due to gravity, whereby a top end of the indicator member is positioned on the top end of the trocar, while a bottom end of the indicator member protrudes from the second tunnel.

41. The safety trocar device as claimed in claim 40, wherein a bottom end of the first indicator member protrudes from the tip of the thrust section; a bottom end of the second indicator member protruding from a circumference of the thrust section.

42. The safety trocar device as claimed in claim 40, wherein each of the top ends of the indicator members has an indicating section.

43. The safety trocar device as claimed in claim 36, further comprising a rotary switch disposed on the top end of the trocar assembly.

44. The safety trocar device as claimed in claim 36, wherein the pulling/lifting section has two hook ears spaced from each other by a certain distance for hooking a body.

45. The safety trocar device as claimed in claim 36, further comprising a locating member disposed between the elevation arm and the bracket body, whereby when the elevation arm is moved upward to the suspension arm, the locating member serves to maintain the height of the elevation arm.
